(19) United States
(12) Patent Application Publication
(10) Pub. No.: US 2001/0003147 A1
(43) Pub. Date: Jun. 7, 2001

(54) Apparatus for measuring musculus orbicularis oris power

(76) Inventors: Shunichi Yokoyama, Tokyo (JP); Yasuhiro Tashiro, Tokyo (JP)

Correspondence Address: Morris Liss, Pollock, Vande Sande & Amernick, R.L.L.P., P.O. Box 19088, Washington, DC, 20036-3425, US

(21) Appl. No.: 09/727,697

(22) Filed: Dec. 4, 2000

(30) Foreign Application Priority Data
Dec. 3, 1999 (JP) 344680

(51) Int. Cl. A61B 5/103

(52) U.S. Cl. 600/590; 600/587

(57) ABSTRACT

An musculus orbicularis oris power measuring apparatus for quantitatively determining the power of the musculus orbicularis oris around the human mouth is provided. A force for closing the mouth lips is detected as an electric output signal by a load sensor shaped so as to be held between the lips. A maximum value of the load force as detected by the load sensor is sensed by a peak hold circuit and stored therein. The thus stored value is indicated either in an analog form or in a digital form by a peak value indicator.

### BACKGROUND OF THE INVENTION

1. Field of the Invention

This invention relates to a mouth muscle force measuring apparatus and more particularly to apparatus for measuring the musclus orbicularis oris power or orbicular muscle power of mouth.

2. Description of the Related Art

Human beings, whoever they may be, will begin to age when they exceed a certain age, and if things should get worse, will deteriorate in brain function and begin to dote to dementia. Recent researches have shown that moving the lips actively results in increasing the cerebral blood flow. Further, it is being elucidated that deterioration in the brain function may be retarded by training the musculus orbicularis oris which is an orbicular muscle around mouth.

Various devices for training the musculus orbicularis oris have also been developed. An example of stretch devices designed for training the musculus orbicularis oris is illustrated in FIGS. 18-20. Thie stretch device is generally designated by the reference number 1. The stretch device 1 is formed of a resin material capable of elastic deformation and is generally shaped so as to conform with the shape of the human mouth and lips. The device is configured such that with the opposed widened sections 2 of the device held in the subject's or examinee's mouth while his or her upper and lower lips are inserted in the upper and lower channels 3 of the device, respectively, so that the subject may repeat the action of closing his or her lips. The closing action is effected against the repulsion generated by the resin material as it is deformed whereby the musculus orbicularis oris is gradually trained. The relation between the development (restoration) of the musculus orbicularis oris power and the degree of rehabilitation of the brain function is also under study.

### BRIEF SUMMARY OF THE INVENTION

Instruments for measuring the phenomenon of the cerebral blood flow increasing while the lips are in motion are already in existence and have been put in practical use. However, there has not yet been developed means for determining how much the muscle force or musculus orbicularis oris power (which is also called muscle force of mouth lips) of an individual and whether the muscle power is in the tendency to increase or to decline. Consequently, as matters stand, it is not possible to quantitatively determine the relation between the musculus orbicularis oris power and the brain function.

Since it is somewhat presumed from the studies conducted heretofore that there is some correlation between a decline in the musculus orbicularis oris power and progress of the dotage, it is desirable for individuals to train their musculus orbicularis oris as much as possible and to have means for apprehending any tendency of their musculus orbicularis oris power to decline as early as possible.

Accordingly, it is a first object of this invention to provide a musculus orbicularis oris power measuring apparatus for use to quantitatively determine whether the musculus orbicularis oris power is in the tendency to decline or increase and to quantitatively determine the relation between the musculus orbicularis oris power and the brain function.

It is a second object of this invention to provide an musculus orbicularis oris power measuring apparatus for providing for readily measuring the musculus orbicularis oris power at individual homes and for an individual to apprehend a decline of the musculus orbicularis oris power as early as possible and further providing for measuring and ascertaining how much the musculus orbicularis oris power has been recovered so far during the training.

According to this invention, an musculus orbicularis oris power measuring apparatus is provided which comprises:

a load sensor configured so that a subject may hold the sensor between his or her mouth lips for measuring the magnitude (load value) of the force of the musculus orbicularis oris for closing the lips;

peak hold means for detecting a maximum of the load value sensed by the load sensor and storing the maximum value; and

an indicator for indicating the maximum value stored in the peak hold means.

As set forth in claim 2, the load sensor and a measuring instrument proper accommodating the indicator and the peak hold means may be separately constructed and interconnected by means of a cable. Or alternatively, as set forth in claim 3, the load sensor may be incorporated in a portion of the measuring instrument proper accommodating the indicator and the peak hold means so that the load sensor and the measuring instrument proper are integrated together.

In addition, as set forth in claim 4, the indicator may be an analog pointer type indicator and the peak hold means may comprise an analog peak hold circuit.

In another embodiment, as set forth in claim 5, the indicator may be a digital indicator and the peak hold means may also comprise a digital circuit.

The load sensor, as set forth in claim 6, may comprise an elastically deformable elastic or resilient member and a strain sensor affixed to the elastically deformable elastic member.

Alternatively, the load sensor, as set forth in claim 7, may comprise an elastically deformable elastic member and a displacement detector for measuring the amount of deformation of the elastic member.

In a still alternative form, the load sensor, as set forth in claim 8, may comprise a pressure-sensing sensor.

According to this invention, a squeezing force by the lips, which is a load force by the force of the musculus orbicularis oris, is applied to the load sensor held between the lips so that the load sensor may measure the squeezing force (load value), a maximum value of which is stored by the peak hold means and then indicated as the peak held value on the indicator. It will thus be appreciated that this apparatus is capable of reliably measuring even an instantaneously produced force.

Accordingly, this apparatus allows for quantitatively measuring the musculus orbicularis oris power and hence definitely determining by continuously conducting the measurement whether the muscle power is in the tendency to decline or to increase. It also allows for quantitatively comparing the musculus orbicularis oris power with the brain function and analyzing the correlation between the musculus orbicularis oris power and the brain function.

The term “elastically deformable elastic member” herein used is intended to mean that the material used for the load sensor in the present invention is required to maintain elasticity under a maximum of the load power exerted by the musculus orbicularis oris. Materials suitable for the load sensor may include metal such as aluminum and stainless steel, for example. However, it is merely for the reason that such metal has a thermal expansion coefficient approximate to that of the strain sensor used so that there is less likeliness that the elastic member and the strain sensor joined together may peel apart, and therefore it is to be understood that this invention is not limited to such exemplification.

### BRIEF DESCRIPTION OF THE DRAWINGS

FIG. 1 is a diagrammatic view illustrating the connection and arrangements of the components of an embodiment of this invention;

FIG. 2 is a representation illustrating the operation of the load sensor used in the embodiment shown in FIG. 1;

FIG. 3A is a front view illustrating another form of the load sensor used in the embodiment shown in FIG. 1;

FIGS. 3B and 3C are a plan view and a front view, respectively showing an alternative form of the load sensor shown in FIG. 3A;

FIG. 4A is a plan view illustrating an example of the combination of the load sensor shown in FIG. 3A and the measuring instrument proper;

FIGS. 4B and 4C are a plan view and a perspective view, respectively illustrating an example of the combination of the load sensor shown in FIGS. 3B and 3C and the measuring instrument proper;

FIG. 5 is a diagrammatic view illustrating the connection and arrangements of the components of an alternative form of the embodiment shown in FIG. 1;

FIG. 6 is a perspective view illustrating an alternative form of the load sensor usable in the present invention;

FIG. 7 is a side view illustrating another alternative form of the load sensor usable in the present invention like that shown in FIG. 6;

FIG. 8 is a perspective view illustrating a still other alternative form of the load sensor usable in the present invention like that shown in FIG. 6;

FIG. 9 is a perspective view illustrating a yet another form of the load sensor usable in the present invention like that shown in FIG. 6;

FIGS. 10A and 10B are plan views illustrating an embodiment of the musculus orbicularis oris power measuring apparatus set forth in claim 3;

FIGS. 11A and 11B are side views of the musculus orbicularis oris power measuring apparatus shown in FIGS. 10A and 10B, respectively;

FIGS. 12A and 12B are plan views illustrating an alternative embodiment of the musculus orbicularis oris power measuring apparatus set forth in claim 3;

FIGS. 13A and 13B are side views of the musculus orbicularis oris power measuring apparatus shown in FIGS. 12A and 12B, respectively;

FIG. 14 is a schematic view illustrating a modified embodiment of this invention;

FIG. 15 is a rear view of the embodiment of FIG. 14 cut away to illustrate the interior construction;

FIG. 16 is a side view of the embodiment of FIG. 14 illustrating the interior construction;

FIG. 17 is a representation illustrating the reset mechanism for use in the embodiment shown in FIG. 14;

FIG. 18 is a front view illustrating an example of the known prior art stretch device;

FIG. 19 is a side view, partly in cross-section, taken along the line 19-19 in FIG. 18; and

FIG. 20 is a perspective view illustrating the stretch device of FIG. 18 being applied to a subject.

### DETAILED DESCRIPTION OF THE INVENTION

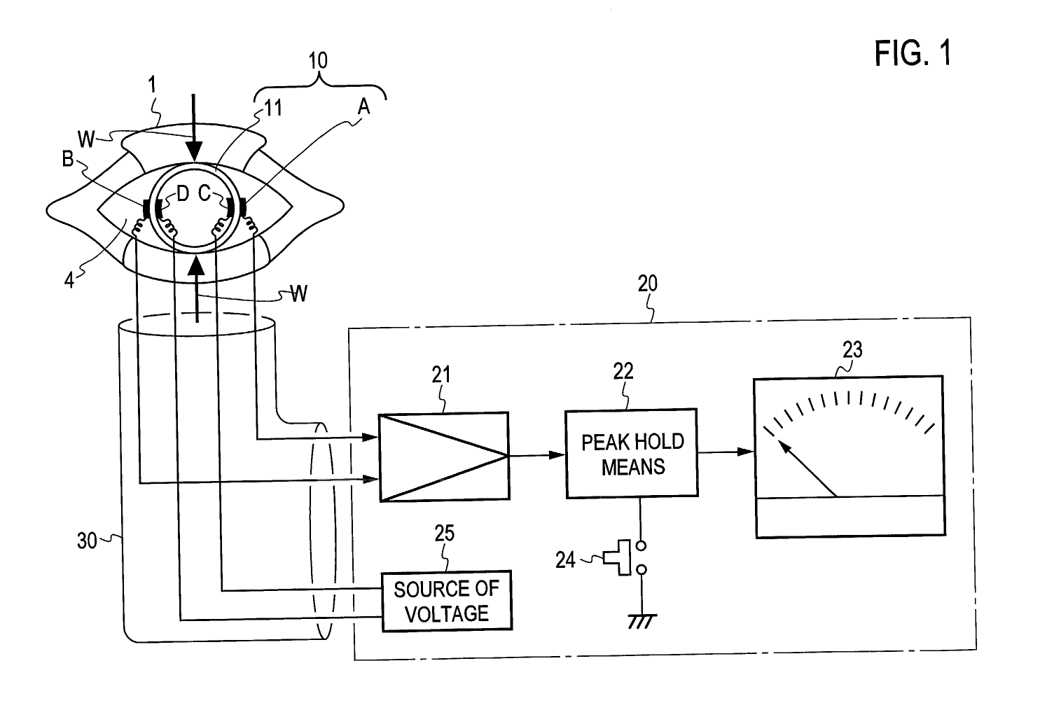

FIG. 1 illustrates one embodiment of the musculus orbicularis oris power measuring apparatus according to this invention. In this embodiment, as set forth in claim 2, the load sensor 10 and the measuring instrument proper 20 are interconnected by means of a cable 30, and the indicator 23 is comprised of an analog indicator as set forth in claim 4 while in this case the load sensor 10 is in the form of a metal ring as set forth in claim 6.

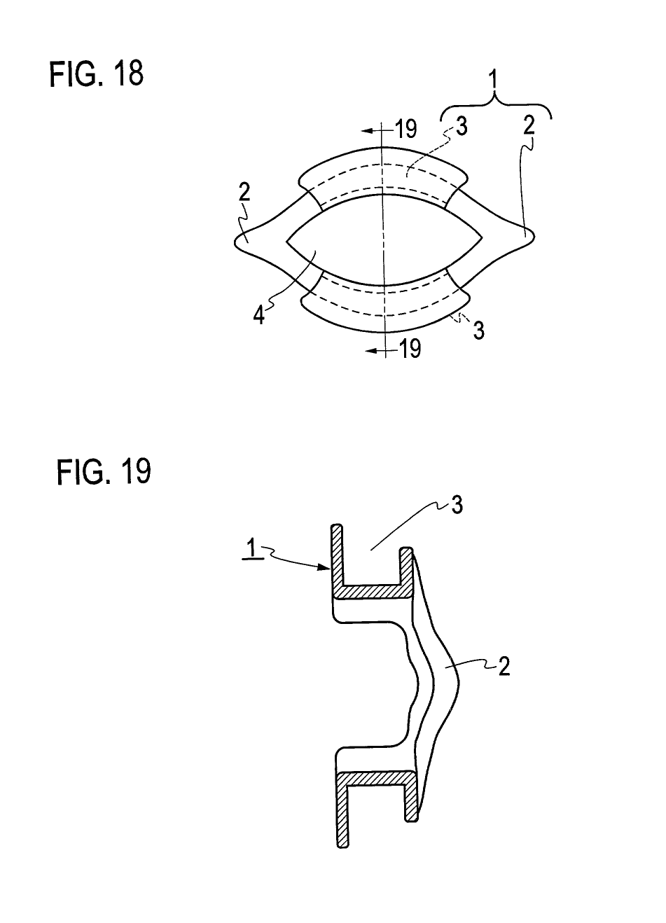

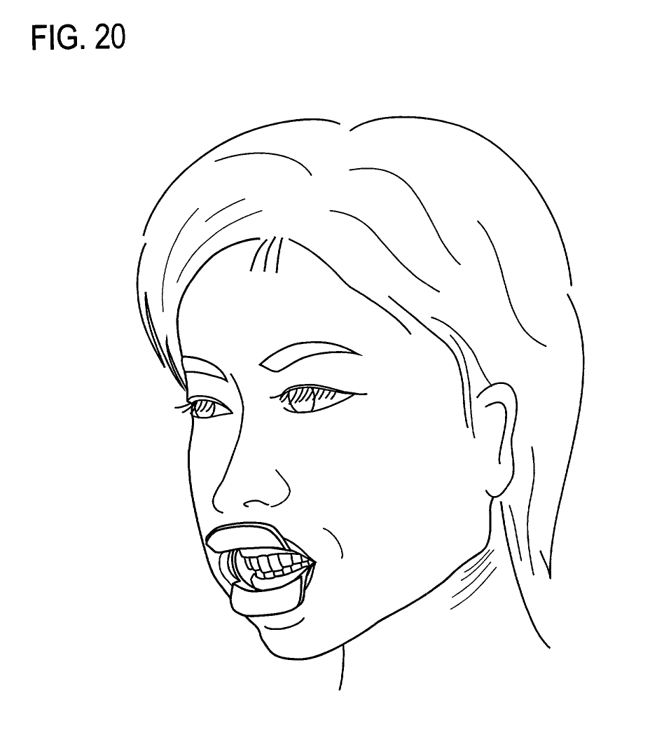

More specifically, this embodiment illustrates the case where the load sensor 10 comprises a metal ring or an elastic member 11 and strain sensors A, B, C and D bonded on or otherwise applied to the outer and inner peripheral side surfaces of the metal ring 11. The metal ring 11 may be constructed of a cylindrical tube of stainless steel or aluminum, for example. The tube may have an axial length in the order of 5-10 mm, a wall thickness of about 1 mm, and a rigidity capable of withstanding a load in the order of maximum 50 N (Newton), where 50 N may be a maximum value of the muscle force exerted by the musculus orbicularis oris. The outer diameter of the ring is set at about 15 mm φ so as to conform to the size of the mouth of a subject so that the ring may be shaped and sized to be inserted in the central opening 4 of the stretch device 1 illustrated in FIG. 18.

Further, it is to be understood that the stretch device 1 is not limited to the type illustrated in FIG. 18, and that alternatively the metal ring 11 may be provided with receptacles adapted to directly receive the mouth lips of a subject for measurement of the musculus orbicularis oris.

The strain sensors A, B, C, D may be in the form of strain sensors known as strain gauge. The strain sensors A, B, C, D may be bonded to the outer and inner peripheral side surfaces of the metal ring 11 in radially aligned opposed locations (aligned on the horizontal line extending through the center of the metal ring as viewed in FIG. 1).

The metal ring 11 is inserted in the stretch device 1 such that the force W of the musculus orbicularis oris is exerted on the metal ring in the direction (vertically as viewed in FIG. 1) orthogonal to the line extending through the locations of the strain sensors A, B, C, D. It is to be noted here that the load sensor 10 and the stretch device 1 are separately handled and that the load sensor 10 is inserted in the stretch device 1 only when the measurement is conducted.

Further, the metal ring need not necessarily be right circular, but be elliptical, and the strain sensors A, B, C, D need not necessarily be bonded to the metal ring at horizontally aligned locations, but at any other desired locations intermediate the horizontal and vertical lines.

In addition, it should be noted that the subject need to hold the metal ring against dislodgement while it is inserted in the stretch device 1 for the measurement. To this end, the metal ring may be configured to be fixed in place by appropriate means although not illustrated.

When the muscle force W (compressive load) of the musculus orbicularis oris is exerted on the metal ring in the vertical direction orthogonal to the line extending through the locations of the strain sensors A, B, C, D, the electrical resistances of the strain sensors A and B affixed to the outer peripheral side surface vary in an increasing sense whereas the resistances of the strain sensors C and D on the inner peripheral side surface vary in a decreasing sense.

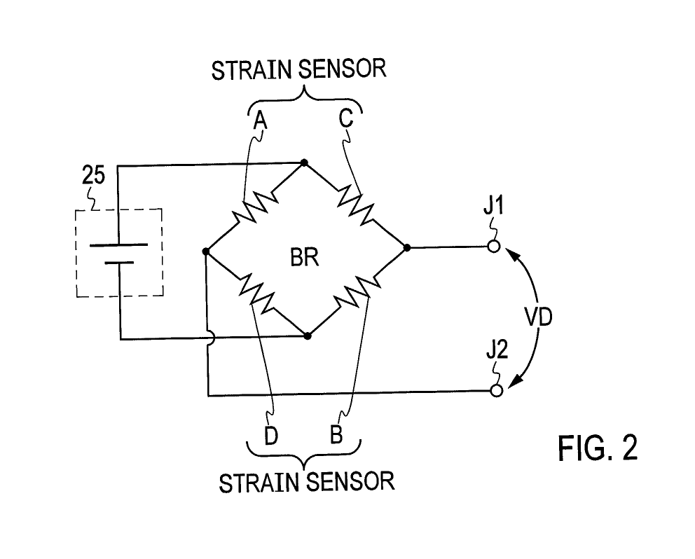

While the connection of the strain sensors A, B, C, D is diagrammatically shown in FIG. 1, specifically they are connected to form a bridge circuit BR as shown in FIG. 2 such that the bridge circuit is in balance under no load. As the resistance of the strain sensors A and B increases while the resistance of the strain sensors C and D decreases with an increase in the load imposed thereon, an output voltage VD corresponding to the change in the resistances of the strain sensors A, B, C, D is generated between the output terminals J1 and J2 of the bridge circuit BR.

As the output voltage VD is generated in proportion to the force W of the musculus orbicularis oris, the voltage VD may be amplified by an amplifier 21 as required, and a peak value of the voltage is held through a peak hold means 22 and then provided to an indicator 23 so that a value corresponding to the force W of the musculus orbicularis oris may be displayed in the indicator 23. By preliminarily calibrating the force W of the musculus orbicularis oris applied to the metal ring 11 and the indication value of the indicator 23, the musculus orbicularis oris power may be read out in units of g (grams) or Kg (kilograms) directly from a value indicated in the indicator 23.

In this embodiment, the peak hold means 22 is illustrated as comprising an analog type peak hold circuit. The conventional principal part of the analog type peak hold circuit is comprised of an unidirectional element (diode) and a capacitor, the circuit arrangement being such that a load sensing signal output through the diode from the amplifier 21 is charged in the capacitor whereby even when the load sensing signal returns to zero subsequently, the maximum voltage remains held in the capacitor. Since this peak hold circuit is a conventional circuit well known in the art, further description is omitted. 24 designates a reset switch for resetting the voltage peak-held in the capacitor to zero. 25 designates a source of voltage for applying a voltage to the load sensor 10.

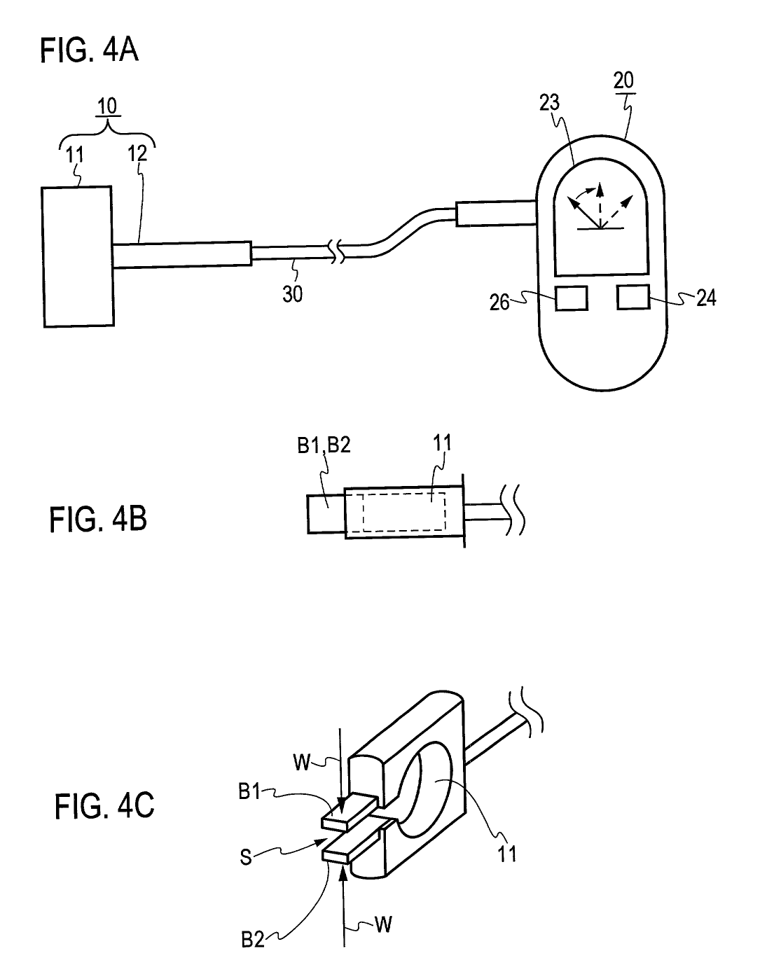

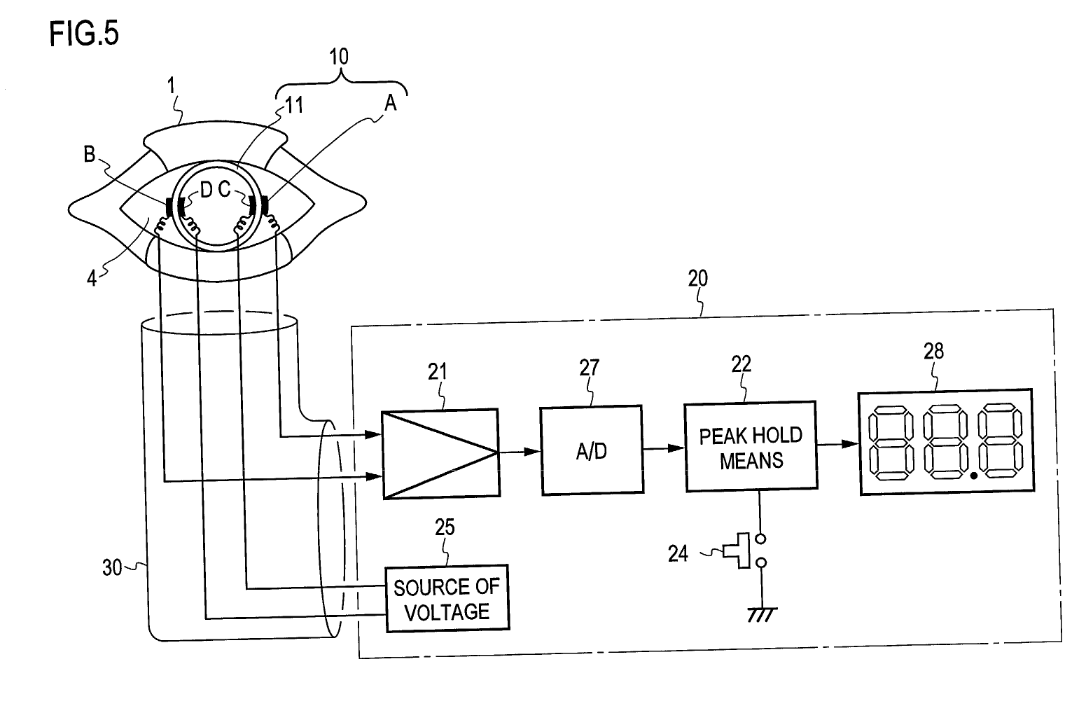

Connected to the other end of the cable 30 is a measuring instrument proper 20. The measuring instrument proper 20 has the analog indication type indicator 23 as shown in FIG. 1, the reset switch 24 for resetting the indication, and a power switch 26 (FIG. 4A) arranged in the front face.

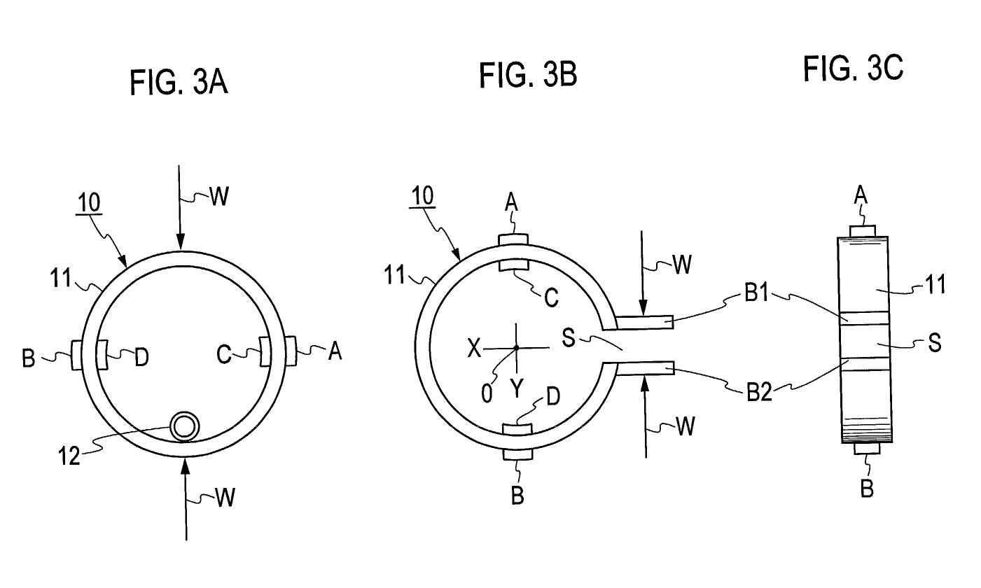

FIGS. 3A and 4A illustrate the basic configuration of the load sensor 10 in which the metal ring 11 is provided with a handle 12 through which the cable 30 is passed to be connected with the strain sensors A, B, C, D attached to the metal ring 11. To this end, the handle 12 is in the form of a pipe through the hollow bore of which the cable 30 is passed to maintain the connection between the cable 30 and the strain sensors A, B, C, D in a reliable condition. The handle 12 is gripped by hand by a person who is engaged in measuring the musculus orbicularis oris power when the person inserts the metal ring 11 of the load sensor 10 into the opening 4 of the stretch device 1 held in the mouth of a subject. Of course, the subject himself or herself may grip the handle and insert the metal ring into the opening of the stretch device.

FIGS. 3B and 3C are a plan view and a front view, respectively showing an alternative form of the load sensor shown in FIG. 3A, and FIGS. 4B and 4C are a plan view and a perspective view, respectively illustrating an example of the combination of the load sensor shown in FIGS. 3B and 3C and the measuring instrument proper. The strain sensors A, B, C, D are bonded to the inner and outer surfaces of the peripheral wall of the metal ring 11 in alignment on the line Y extending vertically through the center ◯ of the metal ring 11 as viewed in FIG. 3B, and the metal ring 11 is formed with a slit S axially extending through the peripheral wall thereof on one side thereof (on the right side as viewed in FIG. 3B) on the line X extending horizontally through the center ◯ of the metal ring 11 as viewed in FIG. 3B. A pair of bill-like protuberances B1 and B2 are secured to the opposed edges of the ring at the slit S thereof so that the load force W may be applied between the protuberances B1 and B2 perpendicularly thereto. It should be appreciated that this modified embodiment renders the strain sensors very sensitive to the load force W to be sensed.

FIG. 5 illustrates an alternative embodiment in which the measuring instrument proper 20 is comprised of a digital circuit. In this configuration as well, there may be provided an amplifier 21, as required, through which the output voltage is amplified to a desired level. The thus amplified voltage corresponding to a load sensing signal is converted by an analog-to-digital (A/D) converter 27 to a digital signal. Then, the load sensing signal thus converted into a digital signal is applied to a peak hold means 22 which is comprised of a digital circuit. The peak hold means 22 stores a maximum value of the load sensing signal applied thereto and the peak-held value is then indicated in an indicator 28 which comprises a digital indicator of the numerical value display type.

The peak hold means 22 comprised of a digital circuit is also provided with a reset switch 24. After the measurement is completed, the operator may operate the reset switch 24 to reset the peak-held value to zero.

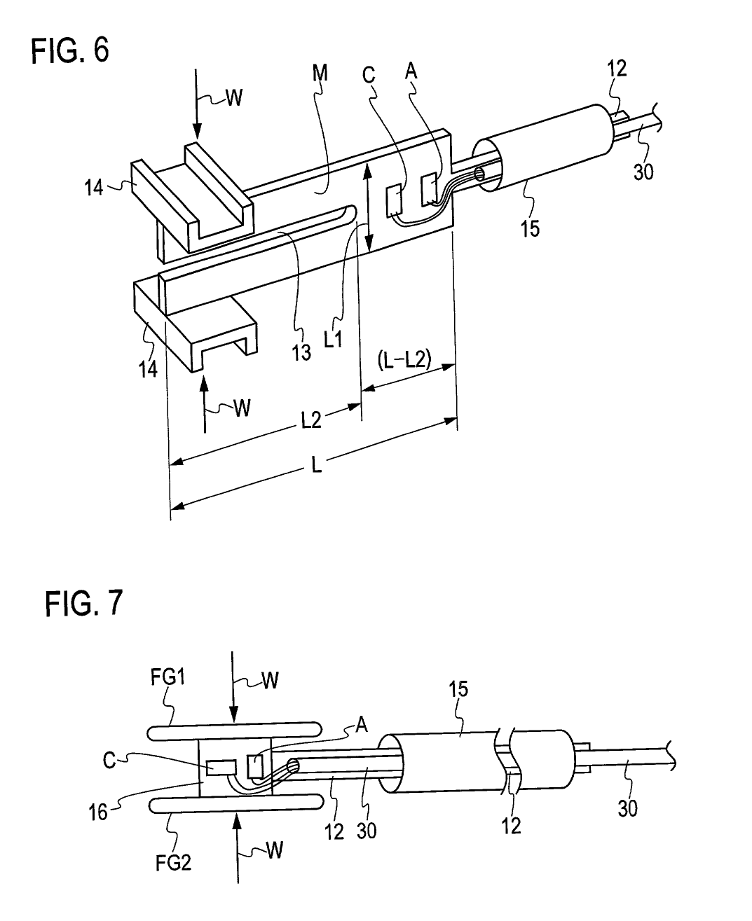

FIGS. 6-9 illustrate various alternative forms of the load sensor 10. In the embodiment shown in FIG. 6, the load sensor comprises a generally rectangular metal plate (such as aluminum plate) M formed with a slit 13 extending longitudinally for a distance L2 from one longitudinal end toward the other end in the middle of the minor side length L1 of the plate, the distance L2 being longer than half the major side length L of the plate, and strain sensors A, B, C, D bonded to the opposite side surfaces of the metal plate within the continuous region (L-L2) where the slit 13 is not formed. While only the strain sensors A and C are shown in FIG. 6, the other strain sensors B and D are bonded to the invisible opposite side surface of the metal plate. Further, it should be noted that the strain sensors A and B are located at positions relatively remote from the slit 13 whereas the strain sensors C and D are located at positions relatively close to the slit 13.

With this construction, when the force W of the musculus orbicularis oris is applied to the metal plate M adjacent the open end of the slit 13, the strain sensors A and B are subjected to strain tending to tension them so that the resistance of the strain sensors A and B varies in an increasing sense whereas the strain sensors C and D are subjected to strain tending to compress them so that the resistance of the strain sensors C and D varies in a decreasing sense.

It will be thus appreciated that in the same manner described with reference to FIG. 2, application of the force W of the musculus orbicularis oris unbalances the bridge circuit BR, so that a load sensing signal corresponding to the force of the musculus orbicularis oris is output from the bridge circuit.

The metal plate M is in the form of a rectangular strip having a width (minor side length) L1 of 15 mm and a length (major side length) L of about 50-80 mm, for example. A pair of receptacles 14 adapted to receive a stretch device 1 as shown in FIGS. 18 and 19 are attached to the metal plate on its opposed upper and lower longitudinal edges adjacent the open end of the slit 13. Further, the metal plate has a handle 12 extending rearwardly from its rear end. The handle 12 may be formed integrally with the metal plate or may be a separate metal member. The cable 30 extends along the handle 12 and is surrounded and clamped together with the handle by a protective sleeve 15 or the like so as to protect the joint between the cable and the strain sensors A, B, C, D. In addition, the metal plate M and the strain sensors A, B, C, D may be covered by a suitable cover (not shown) to prevent contamination from the exterior.

FIG. 7 illustrates still another embodiment of the load sensor 10. In this embodiment, the load sensor comprises an elastically deformable metal rod 16 extending between a pair of opposed spaced flanges FG1 and FG2, and a first pair of strain sensors A and B vertically oriented and bonded to the opposite side surfaces of the metal plate (sensor B is bonded to the back side invisible in the drawing) and a second pair of strain sensors C and D horizontally oriented and bonded to the opposite side surfaces of the metal plate (sensor D is bonded to the back side invisible in the drawing). Since a strain sensor (strain gauge) will act to vary its resistance in response to changes in strain in a longitudinal direction, the strain sensors A and B are subjected to compressive load to vary their resistances in decreasing sense whereas the horizontally disposed strain sensors C and D do not respond to compressive load, and hence do not vary their resistances. It is thus to be understood that in this example as well, when the force W of the musculus orbicularis oris is applied between the flanges FG1 and FG2, the resistances of the strain sensors A and B of the bridge circuit BR shown in FIG. 2 are reduced, resulting in unbalancing the bridge circuit, so that a load sensing signal corresponding to the compressive load may be output from the bridge circuit.

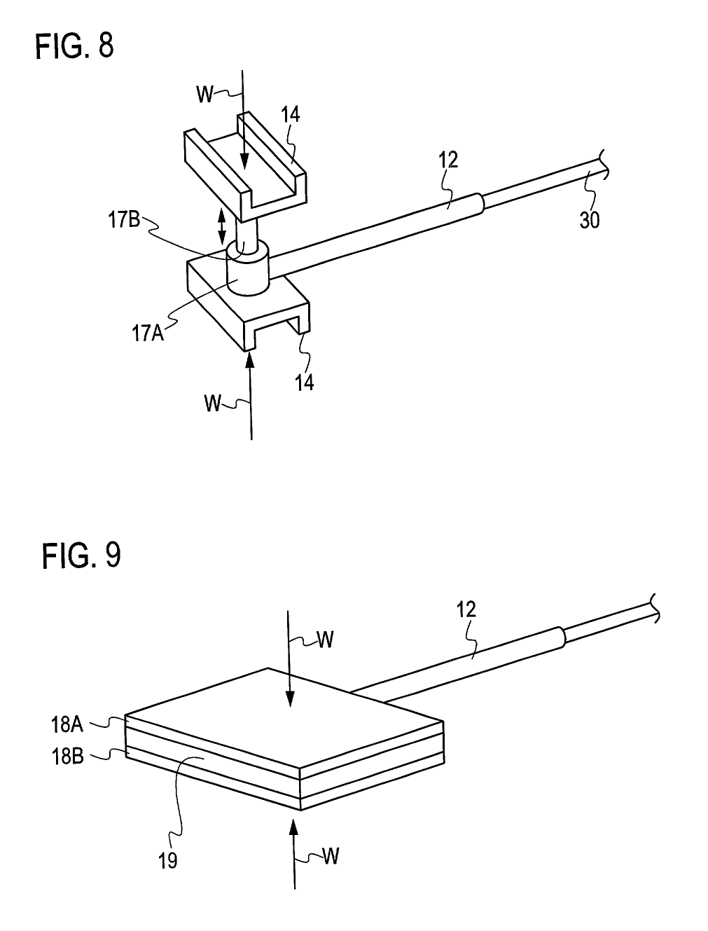

FIG. 8 illustrates yet another embodiment of the load sensor 10. In this embodiment, the load sensor comprises a stationary rod 17A, a movable rod 17B slidably mounted in the interior of the stationary rod 17A, and a differential transducer, the arrangement being such that the amount of deformation of the stretch device 1 as shown in FIGS. 18 and 19 may be measured. In this case, by calibrating the relation between the amount of deformation of the stretch device 1 and the load value, the musculus orbicularis oris power may be accurately measured.

FIG. 9 illustrates yet another embodiment of the load sensor 10. In this embodiment, the load sensor comprises a pair of electrodes 18A and 18B, and a pressure-sensing element 19 sandwiched between the electrodes 18A and 18B, the configuration being such that the force W of the musculus orbicularis oris may be measured by varying the resistance of the pressure-sensing element 19 by applying the force W of the musculus orbicularis oris to the pressure-sensing element 19 to subject the latter to a compressive load.

The pressure-sensing element 19 may be an electrically conductive rubber-made pressure-sensing element, for example. Typically, the pressure-sensing element will vary its electrical resistance in a decreasing sense as it is subjected to a compressive load. The force W of the musculus orbicularis oris may be measured by measuring the change of this resistance. In addition, it should be noted that although a slight voltage is applied between the electrodes 18A and 18B, it does no substantial harm to the human body. However, in order to ensure safety, insulating sheets or the like may be attached to the surfaces of the electrodes 18A, 18B to electrically insulate them.

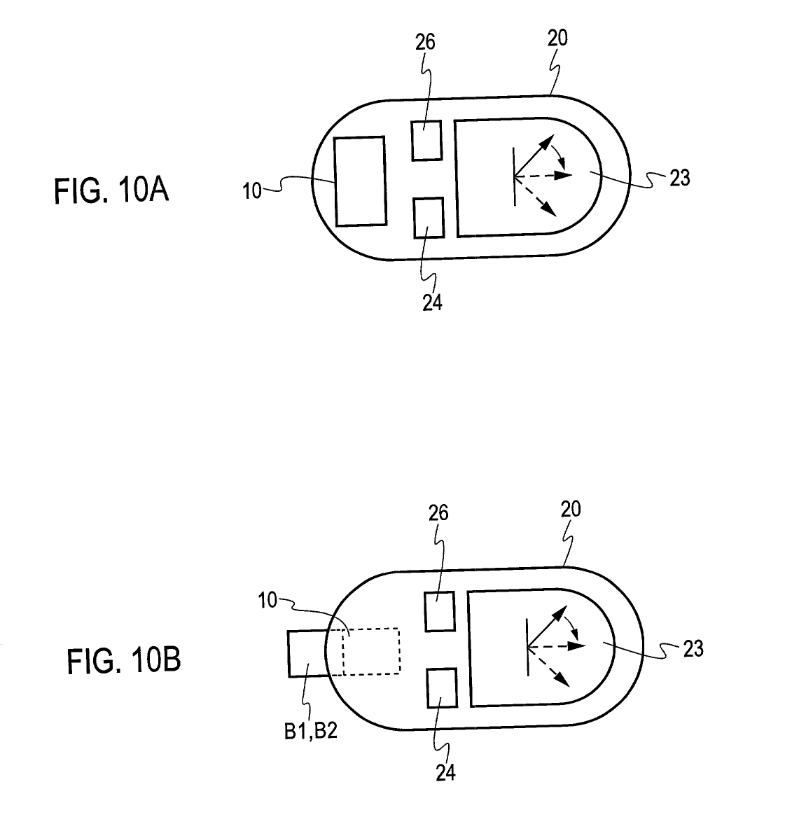

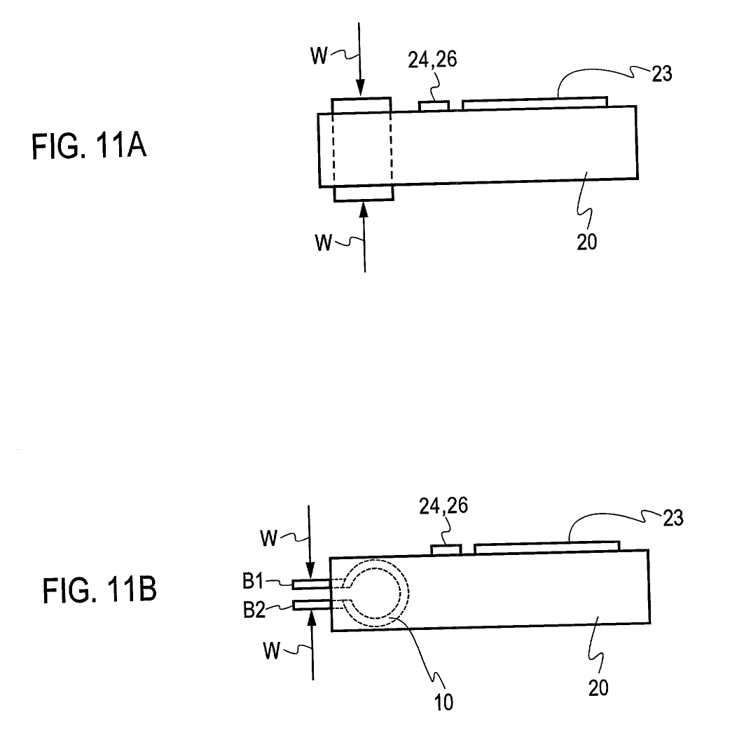

FIGS. 10A and 11A illustrate an embodiment of the musculus orbicularis oris power measuring apparatus as set forth in claim 3. The musculus orbicularis oris power measuring apparatus of this embodiment is characterized in that the measuring instrument proper 20 and the load sensor 10 are integrated together by incorporating the load sensor 10 shown in FIG. 3A into the measuring instrument proper 20 as a part of the latter.

FIGS. 10B and 11B illustrate another embodiment of the musculus orbicularis oris power measuring apparatus as set forth in claim 3. The musculus orbicularis oris power measuring apparatus of this embodiment is characterized in that the measuring instrument proper 20 and the load sensor 10 are integrated together by incorporating the load sensor 10 shown in FIG. 3B into the measuring instrument proper 20 as a part of the latter.

FIGS. 10A, 11A and FIGS. 10B, 11B illustrate embodiments utilizing an analog type indicator 23. The measuring instrument proper 20 is of such size and shape that it can be put on the palm of a hand. The thickness (vertical profile) of the measuring instrument proper, particularly that of the portion in which the load sensor 10 is mounted (FIGS. 10A, 11A) or the thickness between the pair of protuberances B1 and B2 (FIGS. 10A, 11A) are selected at say, 15 mm so as to be inserted in the opening 4 of the stretch device 1 as shown in FIGS. 18 and 19. However, it will be readily understood that the stretch device 1 need not necessarily be used but that the subject's mouth lips may be applied directly against the load sensors or the portions where the load sensors are mounted.

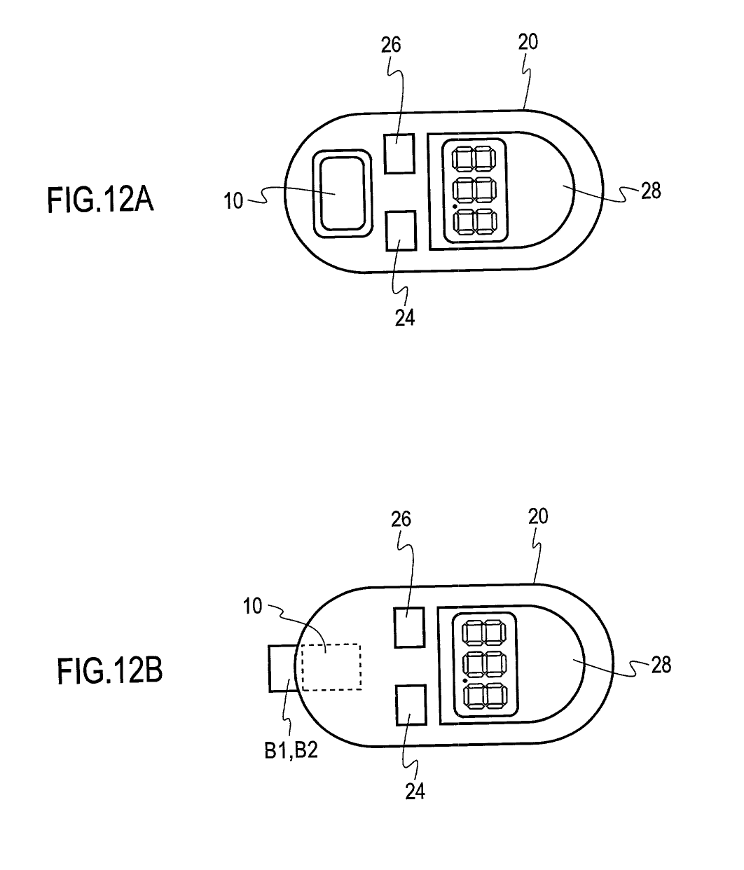

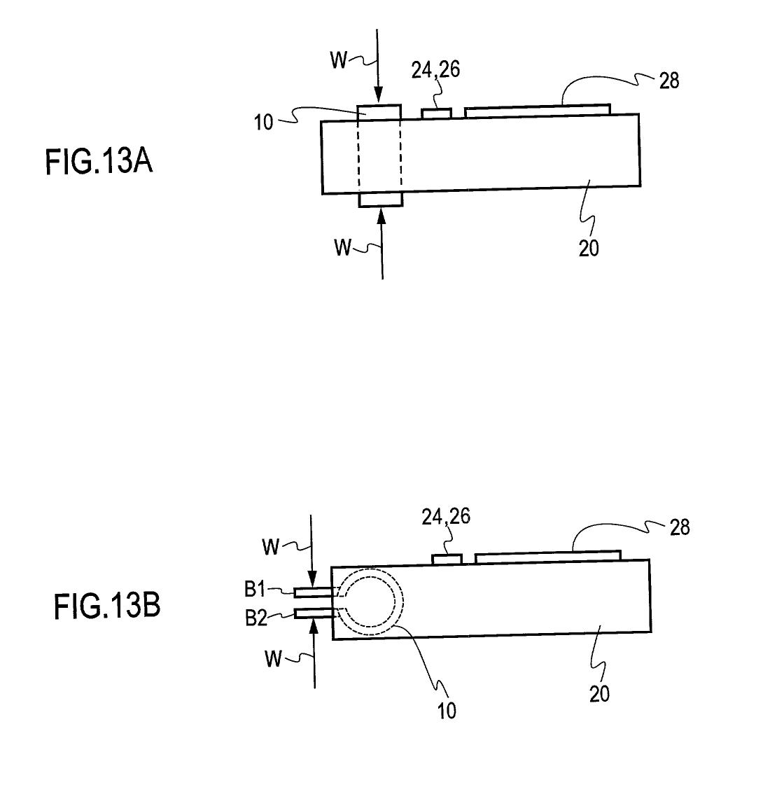

FIGS. 12A, 13A and 12B, 13B illustrate embodiments in which digital indicators 28 are used. Irrespective of whether the indicator is of digital type or analog type, the measuring instrument proper 20 include a reset switch 24 and a power switch 26 to reset the indication and to switch on and off the electric power, respectively. Further, it is to be noted that a selector switch for selecting the ranges of measurements may be provided, if desired, in addition to the reset switch 24 and power switch 26.

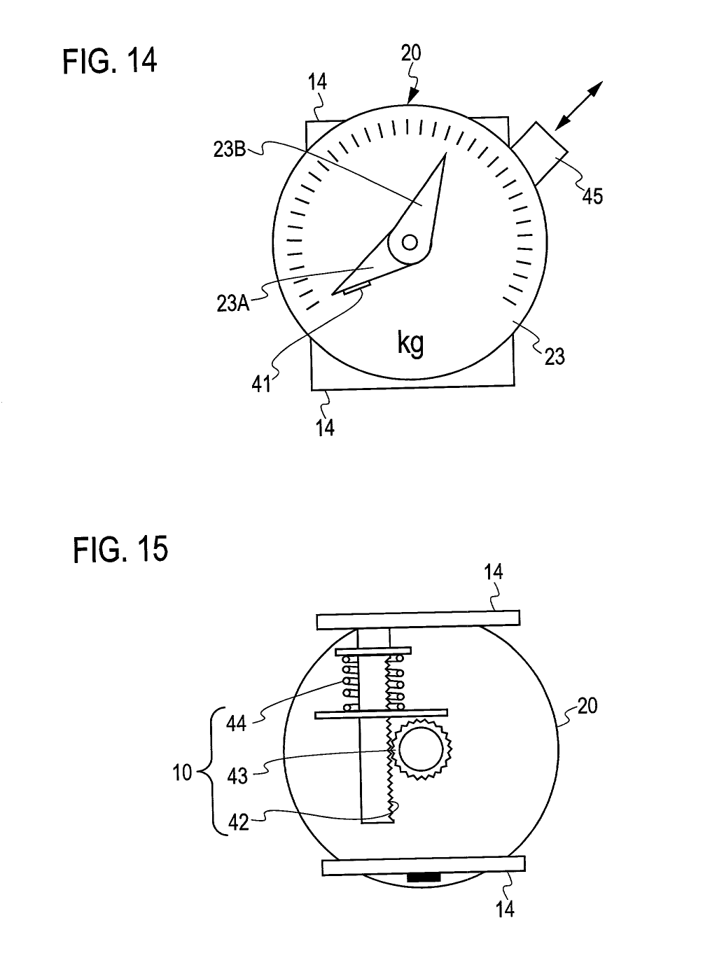

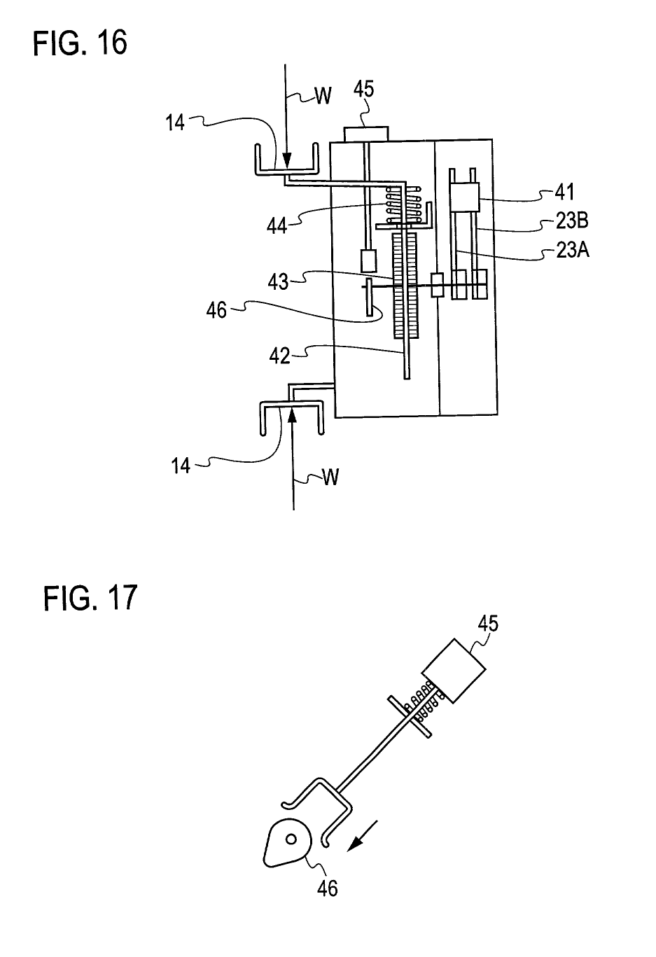

FIGS. 14-17 illustrate a modified embodiment of the musculus orbicularis oris power measuring apparatus of this invention set forth in claim 3. This embodiment represents an instance wherein a spring balance is applied to this invention. The indicator 23 shown in FIG. 14 includes a pointer needle 23A for indicating a varying load and a hold needle 23B comprising a peak hold means.

The pointer needle 23A is adapted to be rotated in accordance with the force W of the musculus orbicularis oris exerted on the receptacles 14 and is provided with a tab 41 engageable with the hold needle 23B so that as the pointer needle 23A is rotated in accordance with the force W of the orbicularis oris muscle exerted on the receptacles 14, the hold needle 23B is also rotated in unison with the pointer needle 23A. When the pointer needle 23A is returned to its zero position after it reaches a maximum value, the hold needle 23B remains at that rotated position to continue indicating the peak value. Consequently, the maximum value of the musculus orbicularis oris power may be measured by taking the reading of the hold needle 23B.

The receptacles 14 are connected to a rack 42 mounted within the measuring instrument proper 20 toward the rear side of the measuring instrument proper 20. The rack 42 is supported for vertical movements in the interior of the measuring instrument proper 20 and is in meshing engagement with a pinion 43, the shaft of which is in turn connected to the pointer needle 23A.

The rack 42 is engaged by a compression spring 44 so as to be biased vertically upwardly by the repulsive force of the spring 44. By preliminarily calibrating this biasing force of the spring and the travel of the rack 42 with reference to a known load value, the power of the musculus orbicularis oris may be read out directly from a reading of the pointer needle 23A.

It is thus to be understood that the spring 44, the rack 42 and the pinion 43 constitute a displacement-sensing type load sensor 10.

In addition, it is seen in FIGS. 14-17 that the measuring instrument proper 20 includes a reset button 45 which is adapted, upon being depressed, to rotate a cam 46 (FIG. 17) secured to a shaft carrying the hold needle 23B (inner shaft extending through the interior of the outer shaft of the pointer needle 23A) in a predetermined direction to thereby reset the hold needle 23B to its zero position.

As discussed above, this invention allows for quantitatively determining the musculus orbicularis oris power and hence quantitatively measuring and recording the relation between the musculus orbicularis oris power and the brain function. It is thus to be appreciated that this invention may be utilized as means for elucidating the relation between the musculus orbicularis oris power and the brain function.

In addition, the configuration as shown in FIGS. 10-17 in which the measuring instrument proper 20 is integrated into the load sensor 10 makes it possible for anyone to easily measure his or her musculus orbicularis oris power. Hence, individuals may apprehend a decline of their musculus orbicularis oris power as early as possible by measuring their own musculus orbicularis oris power at home. It is expected that this invention provides the great advantage that progress of dotage may be arrested by training the musculus orbicularis oris by the use of the stretch device 1, for example.

What is claimed is:

1. An apparatus for measuring musculus orbicularis oris power comprising:
a load sensor configured so as be held between mouth lips of a subject and adapted to transform a force of the subject's musculus orbicularis oris for closing the lips to a load value and output the load value;
peak hold means for detecting a maximum of the load value sensed by said load sensor and storing the maximum value; and
an indicator for indicating the maximum value stored in said peak hold means.

2. The musculus orbicularis oris power measuring apparatus according to claim 1, in which
said peak hold means and said indicator are accommodated in a measuring instrument proper which is formed separately from said load sensor and connected to said load sensor by means of a cable.

3. The musculus orbicularis oris power measuring apparatus according to claim 1, in which said load sensor, said peak hold means and said indicator are accommodated in a measuring instrument proper.

4. The musculus orbicularis oris power measuring apparatus according to any one of claims 1-3, in which said indicator is an analog indicator and said peak hold means comprises an analog peak hold circuit.

5. The musculus orbicularis oris power measuring apparatus according to any one of claims 1-3, in which said indicator is a digital indicator and said peak hold means comprises a digital circuit.

6. The musculus orbicularis oris power measuring apparatus according to any one of claims 1-3, in which said load sensor comprises an elastic member capable of being elastically deformed by a force of the subject's musculus orbicularis oris, and a strain sensor affixed to said elastic member for measuring a strain of said elastic member.

7. The musculus orbicularis oris power measuring apparatus according to any one of claims 1-3, in which said load sensor comprises an elastic member capable of being elastically deformed by a force of the subject's musculus orbicularis oris, and a displacement detector for measuring an amount of deformation of said elastic member.

8. The musculus orbicularis oris power measuring apparatus according to any one of claims 1-3, in which said load sensor comprises a pressure-sensing element.

9. The musculus orbicularis oris power measuring apparatus according to any one of claims 1-3, in which said load sensor comprises a metal ring capable of being elastically deformed by the force of the subject's musculus orbicularis oris, an element affixed to said metal ring and variable in its electrical resistance in accordance with a strain of said metal ring, and a sensor circuit for measuring the variation in the electrical resistance of said element.

10. The musculus orbicularis oris power measuring apparatus according to claim 9, in which said metal ring is formed with a slit axially extending through the peripheral wall thereof and a pair of bill-like protuberances secured to opposed edges of said ring at said slit, whereby when the subject's load force is applied between said protuberances an electrical output signal in accordance with a strain of said metal ring caused by said load is measured.
